SEQUENCE LISTING (73 sequences)

SEQ ID NO: 1 - protein, 141 aa, Homo Sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLKR

SEQ ID NO: 2 - protein, 142 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLKR

SEQ ID NO: 3 - protein, 68 aa, Mus musculus
EPYLQVDFHTEMKEDSDIAFHSRVYFGHWVVMNSRVNGAWQYEVTCHNMPFQDGKPFNLSISVPPDKY

SEQ ID NO: 4 - protein, 135 aa, Homo Sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 5 - protein, 135 aa, Mus musculus
MACGLVASNLNLKPGECLKVRGEVASDAKSFVLNLGKDSNNLCLHFNPRFNAHGDANTIVCNTKEDGTWGTEHREPAFPFQPGSITEVCITFDQADLTIKLPDGHEFKFPNRLNMEAINYMAADGDFKIKCVAFE

SEQ ID NO: 6 - DNA, 598 bp, Homo sapiens
caattcagaagagccacccagaaggagacaacaatgtccctgctacccgtgccatacacagaggctgcctctttgtctactggttctactgtgacaatcaaagggcgaccacttgtctgtttcttgaatgaaccatatctgcaggtggatttccacactgagatgaaggaggaatcagacattgtcttccatttccaagtgtgctttggtcgtcgtgtggtcatgaacagccgtgagtatggggcctggaagcagcaggtggaatccaagaacatgccctttcaggatggccaagaatttgaactgagcatctcagtgctgccagataagtaccaggtaatggtcaatggccaatcctcttacacctttgaccatagaatcaagcctgaggctgtgaagatggtgcaagtgtggagagatatctccctgaccaaatttaatgtcagctatttaaagagataaccagacttcatgttgccaaggaatccctgtctctacgtgaacttgggattccaaagccagctaacagcatgatcttttctcacttcaatccttactcctgctcattaaaacttaatcaaacttcaaaaaaaaaaaa

SEQ ID NO: 7 - DNA, 526 bp, Homo sapiens
atctctctcgggtggagtccttctgacagctggtgcgcctgcccgggaacatcctcctggactcaatcatggcttgtggtctggtcgccagcaacctgaatctcaaacctggagagtgccttcgagtgcgaggcgaggtggctcctgacgctaagagcttcgtgctgaacctgggcaaagacagcaacaacctgtgcctgcacttcaaccctcgcttcaacgcccacggcgacgccaacaccatcgtgtgcaacagcaaggacggcggggcctgggggaccgagcagcgggaggctgtctttcccttccagcctggaagtgttgcagaggtgtgcatcaccttcgaccaggccaacctgaccgtcaagctgccagatggatacgaattcaagttccccaaccgcctcaacctggaggccatcaactacatggcagctgacggtgacttcaagatcaaatgtgtggcctttgactgaaatcagccagcccatggcccccaataaaggcagctgcctctgctcccctg

SEQ ID NO: 8 - protein, 141 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLK

SEQ ID NO: 9 - protein, 140 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYL

SEQ ID NO: 10 - protein, 139 aa, Homo Sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSY

SEQ ID NO: 11 - protein, 138 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVS

SEQ ID NO: 12 - protein, 137 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNV

SEQ ID NO: 13 - protein, 136 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFN

SEQ ID NO: 14 - protein, 135 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKF

SEQ ID NO: 15 - protein, 133 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLT

SEQ ID NO: 16 - protein, 133 aa, Homo sapiens; identical to SEQ ID NO: 15

SEQ ID NO: 17 - protein, 132 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISL

SEQ ID NO: 18 - protein, 131 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDIS

SEQ ID NO: 19 - protein, 130 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDI

SEQ ID NO: 20 - protein, 129 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRD

SEQ ID NO: 21 - protein, 128 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWR

SEQ ID NO: 22 - protein, 128 aa, Homo sapiens; identical to SEQ ID NO: 21

SEQ ID NO: 23 - protein, 127 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVW

SEQ ID NO: 24 - protein, 126 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQV

SEQ ID NO: 25 - protein, 125 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQ

SEQ ID NO: 26 - protein, 124 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMV

SEQ ID NO: 27 - protein, 123 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKM

SEQ ID NO: 28 - protein, 122 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVK

SEQ ID NO: 29 - protein, 121 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAV

SEQ ID NO: 30 - protein, 120 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEA

SEQ ID NO: 31 - protein, 119 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPE

SEQ ID NO: 32 - protein, 118 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKP

SEQ ID NO: 33 - protein, 117 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIK

SEQ ID NO: 34 - protein, 116 aa, Homo sapiens
MSLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRI

SEQ ID NO: 35 - protein, 115 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRI

SEQ ID NO: 36 - protein, 140 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYLK

SEQ ID NO: 37 - protein, 139 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSYL

SEQ ID NO: 38 - protein, 138 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVSY

SEQ ID NO: 39 - protein, 137 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNVS

SEQ ID NO: 40 - protein, 136 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFNV

SEQ ID NO: 41 - protein, 135 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKFN

SEQ ID NO: 42 - protein, 134 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTKF

SEQ ID NO: 43 - protein, 133 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLTK

SEQ ID NO: 44 - protein, 132 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISLT

SEQ ID NO: 45 - protein, 131 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDISL

SEQ ID NO: 46 - protein, 130 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDIS

SEQ ID NO: 47 - protein, 129 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRDI

SEQ ID NO: 48 - protein, 128 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWRD

SEQ ID NO: 49 - protein, 127 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVWR

SEQ ID NO: 50 - protein, 127 aa, Homo sapiens; identical to SEQ ID NO: 49

SEQ ID NO: 51 - protein, 126 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQVW

SEQ ID NO: 52 - protein, 125 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQV

SEQ ID NO: 53 - protein, 124 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMVQ

SEQ ID NO: 54 - protein, 123 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKMV

SEQ ID NO: 55 - protein, 122 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVKM

SEQ ID NO: 56 - protein, 121 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAVK

SEQ ID NO: 57 - protein, 120 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEAV

SEQ ID NO: 58 - protein, 119 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPEA

SEQ ID NO: 59 - protein, 118 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKPE

SEQ ID NO: 60 - protein, 117 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIKP

SEQ ID NO: 61 - protein, 116 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHRIK

SEQ ID NO: 62 - protein, 115 aa, Homo sapiens; identical to SEQ ID NO: 35

SEQ ID NO: 63 - protein, 114 aa, Homo sapiens
SLLPVPYTEAASLSTGSTVTIKGRPLVCFLNEPYLQVDFHTEMKEESDIVFHFQVCFGRRVVMNSREYGAWKQQVESKNMPFQDGQEFELSISVLPDKYQVMVNGQSSYTFDHR

SEQ ID NO: 64 - DNA, 24 bp, Artificial
  synthetic primer
tacccgtgccatacacagaggctg

SEQ ID NO: 65 - DNA, 25 bp, Artificial
  synthetic primer
cttatctggcagcactgagatgctc

SEQ ID NO: 66 - DNA, 21 bp, Artificial
  synthetic primer
gagcgggaaatcgtgcgtgac

SEQ ID NO: 67 - DNA, 21 bp, Artificial
  synthetic primer
gaaggtagtttcgtggatggc

SEQ ID NO: 68 - DNA, 23 bp, Artificial
  synthetic primer
tcgatgctcttagctgagtgtcc

SEQ ID NO: 69 - DNA, 20 bp, Artificial
  synthetic primer
tgatcgtcttcgaacctccg

SEQ ID NO: 70 - DNA, 23 bp, Artificial
  synthetic primer
gattacagggacatctcaggctg

SEQ ID NO: 71 - DNA, 23 bp, Artificial
  synthetic primer
tatctcttctggctgtagggtgg

SEQ ID NO: 72 - DNA, 22 bp, Artificial
  synthetic primer
ctacgccacgctcatccgctgg

SEQ ID NO: 73 - DNA, 23 bp, Artificial
  synthetic primer
gtagggttggaacacctgctggg

(19) United States
(12) Patent Application Publication
(10) Pub. No.: US 2008/0118515 A1
(43) Pub. Date: May 22, 2008

(54) Regulatory T-Cells Containing Galectins for the Therapy and Diagnosis of Diseases

(75) Inventors: Petra Lutter, Bochum (DE); Petra Weingarten, Dortmund (DE); Christoph Huels, Herne (DE); Hemut E. Meyer, Recklinghausen (DE); Edgar Schmitt, Mainz (DE); Helmut Jonuleit, Ginsheim-Gustavsburg (DE)

Correspondence Address: CONNOLLY BOVE LODGE & HUTZ, LLP, P O BOX 2207, WILMINGTON, DE, 19899, US

(73) Assignee: PROTAGEN AG, Dortmund (DE)

(21) Appl. No.: 10/564,588

(22) PCT Filed: Jul. 15, 2004
(86) PCT No.: PCT/EP04/07890

§ 371 Date: Feb. 10, 2006

(30) Foreign Application Priority Data
Jul. 15, 2003 (DE) 103 33 406.8

(51) Int. Cl. A61K 39/395; C12N 5/08; C07K 16/28

(52) U.S. Cl. 424/144.1; 530/388.22; 435/372

(57) ABSTRACT

The invention relates to regulator T-cells containing galectins, in particular, the use thereof as markers and for the therapy and diagnosis of diseases, in particular, of allergies and autoimmune diseases, in particular rheumatoid arthritis, multiple sclerosis or Crohn's disease, chronic inflammation, asthma, immune deficiency diseases, AIDS, transplant rejection, cancer diseases and diabetes.

The present invention relates to regulatory T cells containing galectins, in particular to their use as markers and for the therapy and diagnosis of diseases, in particular allergies, autoimmune diseases, in particular rheumatoid arthritis, multiple sclerosis or Crohn's disease, chronic inflammation, asthma, immunodeficiency diseases, AIDS, transplant rejection and cancer diseases as well as diabetes.

The invention furthermore relates to suitable binding agents and to a test system (diagnostic agent).

While the immune system is able to differentiate between foreign proteins and structures of its own body, it is also able to distinguish between harmless and pathogenic antigens and consequently avoid unnecessary and autoaggressive immune responses. The ability to maintain immunological tolerance towards endogenous structures while at the same time developing protective immune responses against pathogens is essentially based on forming antigen-specific effector cells for immune defense and forming antigen-specific suppressor cells for preserving immunological tolerance.

Sakaguchi et al. were the first to describe a subpopulation of CD4⁺ T helper cells which are characterized by constitutively expressing the α chain of the IL-2 receptor (CD25), which is essential for controlling autoagressive immune responses in mice (Sakaguchi, S., Sakaguchi, N., Asano, M., Itoh, M., and Toda, M. (1995) Immunologic self-tolerance maintained by activated T cells expressing IL-2 receptor alpha-chains (CD25). Breakdown of a single mechanism of self-tolerance causes various autoimmune diseases. J. Immunol. 155, 1151-1164). By now, these CD4⁺ CD25⁺ T cells have been identified in a variety of species, including humans, as CD25+ regulatory T cells (termed Treg for short in that which follows; characterized by coexpression of the surface proteins CD4+ and CD25+) which, as a resident population, represent 5-10% of the human peripheral CD4⁺ T cells. When freshly isolated, CD25⁺ Tregs are anergic, i.e. while they do not proliferate following allogenic or polyclonal stimulation, they suppress the proliferation of, and cytokine formation by, conventional CD4⁺ and CD8⁺ T cells. While this suppression is cell-contact-dependent and activation-dependent, it is antigen-nonspecific [Jonuleit, H., Schmitt, E., Stassen, M., Tuettenberg, A., Knop, J., and Enk, A. H. (2001) Identification and functional characterization of human CD4(+) (CD25(+) T cells with regulatory properties isolated from peripheral blood. J. Exp. Med. 193, 1285-1294; Dieckmann, D., Plottner, H., Berchtold, S., Berger, T., and Schuler, G. (2001) Ex vivo isolation and characterization of CD4(+)CD25(+) T cells with regulatory properties from human blood. J. Exp. Med. 193, 1303-1310, Ng, W. F., Duggan, P. J., Ponchel, F., Matarese, G., Lombardi, G., Edwards, A. D., Issacs, J. D., and Lechler, R. I. (2001) Human CD4+ CD25+ cells: a naturally occurring population of regulatory T cells, Blood 98, 2736-2744; Seddon, B. and Mason, D. (2000) The third function of the thymus. Immunol. Today 21, 95-99; Seddon, B. and Mason, D. (1999) Peripheral autoantigen induces regulatory T cells that prevent autoimmunity. J. Exp. Med. 189, 877-882; Thornton, A. M. and Shevach, E. M. (1998) CD4+ CD25+ immunoregulatory T cells suppress polyclonal T cell activation in vitro by inhibiting interleukin 2 production. J. Exp. Med. 188, 287-296; Suri-Payer, E., Amar, A. Z., Thornton, A. M., and Shevach, E. M. (1998) CD4+ CD25+ T cells inhibit both the induction and effector function of autoreactive T cells and represent a unique lineage of immunoregulatory cells. J. Immunol. 160, 1212-1219; Piccirillo, C. A., and Shevach, E. M. (2001) Cutting Edge: control of CD8+ T cell activation by CD4+ CD25+ immunoregulatory cells. J. Immunol. 167, 1137-1140].

While depleting the Tregs in vivo results in a number of autoimmune diseases, it also results in improved defense against tumors (Sakaguchi (see above)). This finding supports the thesis that the Tregs have an ambivalent function. While they prevent the development of an autoagressive immune reaction on the one hand, they on the other hand at the same time impede an effective defense against tumors since tumor cells represent the immunological “self”, inter alia, and Tregs therefore stop them being eliminated by effector T cells. Increasing the suppressive function of Tregs is regarded as being helpful for treating autoimmune diseases, in particular, while transiently inhibiting their suppressive properties can support the defense against tumors.

The fact that the suppressive properties are cell-contact-dependent makes it clear that Treg-specific molecules (markers and targets), in particular, have a decisive influence on the functionality of the cells and form the basis for selectively exploiting these properties for therapeutic and diagnostic purposes in the field of allergies, autoimmune diseases, chronic inflammation, immunodeficiency diseases, transplant rejection and cancer diseases as well as AIDS and diabetes.

Proteome analysis was used to selectively investigate the protein composition of the individual T cell subpopulations, in particular the Treg subpopulations (that is CD4⁺ CD25⁺ and CD4⁺ CD25⁺β7⁺ subpopulations) and specifically identify Treg-inherent proteins.

It was surprisingly possible to use proteome analysis to identify β-galactosidase-binding proteins (termed galectins for short in that which follows) such as galectin-1 and galectin-10 (what is termed Charcot-Leyden crystal (CLC) protein).

Galectins are described, for example, in Ni et al. WO98/015624 A1 and Akerman et al. U.S. Pat. No. 5,242,807. However, these documents do not report the specific suitability of the galectins for manipulating and modifying Tregs.

The invention therefore relates to Tregs containing galectins and their isolation. In this connection, galectins in Tregs are suitable markers or targets.

Within the context of this invention, “Tregs” is understood as meaning T cell subpopulations which are of human origin or can be derived from mammals. However, the Treg-CD4⁺ CD25⁺ and Treg-CD4⁺ CD25⁺β7⁺ subpopulations are preferred in accordance with the invention. “Isolated Tregs” are cells which are ex-vivo (outside the living body) and, where appropriate, separated from other T cells. It is also possible to enrich Treg cells which contain galectin by means of isolation (see examples).

The term “native Tregs” describes Tregs which are to be found “in-vivo” (within the living body), e.g. in human blood or thymus or in the mammalian body.

Within the meaning of this invention, “galectins” are proteins having the function of a β-galactosidase binding protein, that is galectins such as galectins 1-14, as human galectin or as a homologous protein from humans or mammals. However, preference is given, according to the invention, to galectin 1 or 10, in particular in accordance with one of the sequences SEQ ID Nos. 1-5. Furthermore, as SEQ ID No. 1 or SEQ ID No. 2, galectin 10 can occur in its isoforms, specifically: a.) apparent molecular weight of 14 kDa and a pI of 6.7, b.) apparent molecular weight of 13.5 kDa and a pI of 5.9, c.) apparent molecular weight of 13 kDa and a Pi of 5.9.

In this connection, the isoforms a.), b.) and c.) can also be present in a truncated form and be acetylated, where appropriate, in accordance with the sequences SEQ ID Nos. 8-64.

The galectins according to the invention can also be modified, e.g. by means of posttranslational modifications such as glycosylation.

WO 98/015624 A1 gives examples of galectins, and galectin 10 is disclosed in Ackerman et al. U.S. Pat. No. 5,242,807. These galectins are included in accordance with the invention.

In another embodiment, the Tregs according to the invention containing galectins are recombinantly altered to the effect that they contain an amino acid sequence according to the invention, preferably SEQ ID No. 1 and SEQ ID No. 2 or SEQ ID No. 4, or a nucleic acid sequence according to the invention, preferably SEQ ID No. 6 or SEQ ID No. 7.

The invention therefore also relates to the amino acid sequences SEQ ID Nos. 1-5 or polypeptides or proteins and their encoding nucleic acid sequences. In particular, SEQ ID No. 1 or SEQ ID No. 2 (galectin 10) only exhibits an agreement of 60% with corresponding sequences specified in WO 98/015624 A1. This is the result of the specific origin of the Tregs according to the invention.

The invention therefore also relates to amino acid sequences (polypeptides or proteins) which exhibit a sequence identify or homology of 70% or more, preferably of 80% or more, particular preferably of 90-95% or more, with SEQ ID No. 1 or SEQ ID No. 2. Those analogous amino acid sequences which, because of the substitution of one or more amino acid(s) in these sequences, nevertheless ensure the desired function of a galectin are likewise included.

Another embodiment likewise relates to fusion proteins which contain an amino acid sequence according to the invention, or a said galectin, as a constituent sequence. Examples of recombinant fusion proteins are given in EP 282 042 B1 (His tag).

The invention furthermore relates to nucleic acids which encode a galectin, preferably a galectin which can be obtained from a Treg, or encode the amino acid sequences according to the invention.

In particular, the nucleic acids according to the invention can be a nucleic acid sequence as depicted in SEQ ID No. 6, encoding SEQ ID No. 1 or SEQ ID No. 2 (galectin 10), or a nucleic acid sequence as depicted in SEQ ID No. 7, encoding SEQ ID No. 4 (galectin 1).

In another preferred embodiment, the nucleic acid according to the invention contains one or more noncoding sequences and/or a poly(A) sequence, one or more recognition sequences and, if required, one or more potential N-glycosylation sites. The noncoding sequences are regulatory sequences, such as promoter or enhancer sequences, for the controlled expression of the encoding gene containing the nucleic acids according to the invention. Furthermore, these nucleic acids can be the subject of customary expression vectors, customary host cells or customary vectors for gene therapy (e.g. J. Sambrook, E. F. Fritsch, T. Maniatis (1989), Molecular cloning: A laboratory manual, 2nd Edition, Cold Spring Harbor Laboratory Press, Cold Spring Harbor, USA or Ausubel, “Current Protocols in Molecular Biology”, Green Publishing Associates and Wiley Interscience, N.Y. (1989)).

The term “nucleic acid” (synonym: polynucleotide) has the meaning of DNA or RNA or chemical analogs and the like.

The galectins according to the invention can secrete and bind to membrane-located proteins on Tregs or effector cells. In addition to this, they can crosslink these membrane-located proteins and therefore influence and regulate their functions. This property can be used in accordance with the invention in order to influence the interaction between Tregs and T effector cells, e.g. for the purpose of treating diseases which are connected with Tregs or an effector cell.

In addition, the galectins according to the invention can be present in the cytosol of the Tregs. The invention therefore relates to such Tregs, with at least one galectin being secreted, being located in the membrane or being presented on the surface or in the cytosol.

Recombinant methods can be used to concentrate at least one galectin in the Treg or on the surface of the Tregs. In order to do this, an amino acid sequence or nucleic acid according to the invention can be introduced into the Treg.

In another embodiment, the novel “Tregs containing galectins” can be recombinantly altered such that they then contain an amino acid sequence according to the invention, preferably SEQ. ID No. 1 or SEQ ID No. 2 or SEQ ID No. 4, or a nucleic acid sequence according to the invention, preferably SEQ ID No. 6 or SEQ ID No. 7.

The invention also relates to agents which bind to at least one isolated regulatory T cell or native regulatory T cell which contains at least one galectin. The binding agents can be selected, without this being limiting, from the group: inhibitor, agonist, antagonist, probe, antibody or immunomodulator.

The binding agents can also induce a signal, such as a color reaction or radioactive label, which is sufficient for identifying and modifying a Treg which contains galectins. The binding agent can therefore be a “probe”. In the widest sense, the binding agent is therefore also, in accordance with the invention, an addressed molecule which binds to a suitable signal-mediating receptor on Tregs containing galectin and generates a feedback on the basis of the galectin which is present in the Treg.

For example, an inhibitor or modulator can be used to advantageously concentrate galectins in Tregs. It is likewise possible for example, to use a probe to identify other Treg cells which contain galectins. An example of such a probe is an antibody which specifically recognizes one or more epitopes which are present on the amino acid sequences according to the invention (e.g. SEQ ID No. 1 or SEQ ID No. 2) or galectins (preparation adapted, for example, from Köhler).

In addition, the binding agent according to the invention can contain one or more epitopes, with one or more epitopes crosslinking with galectins and one or more epitopes crosslinking with surface proteins on Tregs or effector cells, in particular possessing surface proteins such as, for example and without limitation, CD25, CD44, CD45, GITR, CTLA-4 or Fox P3.

From a functional point of view, the binding agents have the function of activating or inactivating the isolated Treg or native Treg containing at least one galectin.

The Tregs containing galectins or binding agents are therefore suitable for being used as pharmaceuticals, preferably for the treatment and therapy of diseases, specifically allergies, autoimmune diseases, in particular rheumatoid arthritis, multiple sclerosis or Crohn's disease, chronic inflammation, asthma, immunodeficiency diseases, AIDS, transplant rejection and cancer diseases as well as diabetes. The autoimmune diseases are in particular those selected from the group: alopecia greata, Bechterew's disease, antiphospholipid syndrome, Addison's disease, Behcet's disease, sprue celiac disease, chronic fatigue immune dysfunction syndrome (CFIDS), polyneuropathy, Churg-Strauss syndrome (granulomatosis), CREST syndrome (Raynaud's syndrome), cold agglutinin disease, cryoglobulinemia, fibromyalgia, fibromyositis, Basedow's disease, Guillain-Barré syndrome, idiopathic pulmonary fibrosis, idiopathic thrombocytopenia, IgA nephropathy, lichen planus, Ménière's disease, polyarteritis nodosa, polychondritis, polyglandular syndrome, polymyalgia rheumatica, primary agammaglobulinemia, biliary cirrhosis, psoriasis, Reiter's disease, sarcoidosis, Sjögren's disease, Takayasu arteritis, vasculitis, vitiligo and Wegener's granulomatosis.

Isolated Tregs containing galectins, which have been modified in accordance with the invention, can be administered to the body which is to be treated. In the second place, adequate doses of suitable binding agents can be administered to the patient. For this purpose, the Tregs containing galectins and/or binding agents can be formulated together with other auxiliary substances, where appropriate.

The invention also relates to the use of the galectins in Tregs as markers or targets.

In particular, the galectins can serve as targets for manipulating or modulating the suppressive properties of a Treg. This can be effected, for example, by means of a binding agent or a substance. Furthermore, the binding agent or the substance can be an inhibitor which stops, inhibits or promotes the expression of the galectin. The Treg-specific galectins can also serve as markers for identifying Tregs which have (elevated) suppressive properties.

The invention also relates to a test system, comprising at least one binding agent and at least one Treg containing galectins, for identifying suitable binding agents or Tregs, preferably those possessing elevated suppressive properties.

The invention therefore also relates to a test system which comprises at least one Treg containing galectins and at least one target cell, in particular T cell, B cell, macrophage, predendritic cell, dendritic cell, embryonic cell and/or fibroblast which is/are incubated with at least one Treg for the in-vitro detection of suppressive properties, in particular suppression of the cellular immune response of effector cells of the immune system, in particular B cells, NK cells, preferably T cells and T helper cells.

The cellular immune response of the target cells can be examined in the test system according to the invention on the basis of the special cell-contact-dependent suppressive properties of the Tregs containing galectins.

An immune response can be detected, for example, by the synthesis of cytokines such as gamma interferon or interleukins. In this test system, the appropriate cytokine accumulates intracellularly and can be detected by way of fluorescence-coupled antibodies (e.g. ELISA). In addition, by means of the expression of surface molecules, lysis of the target cell or cell proliferation. The proportion of the immune cells which can be stimulated or not stimulated or activated or inactivated can be determined in a FACS (fluorescent activated cell sorter). Other detection methods, without them being limiting, are cytokine assay, ELISPOT, proliferation tests and 51Cr-release tests (in this regard, see, in a general manner: Current Protocols of Immunology (1999), Coligan J. E., Kruisbeek A. M., Marguiles D. H., Shevach E. M. and Strober W., John Wiley & Sons). However, nonradioactive detection methods are preferred.

In another embodiment, the effector cells are mammalian cells, in particular human or murine cells, or an immune cell line and/or a cultured primary immune cell.

In another embodiment, at least one further substance which can induce an immune response, such as proteins, epitopes, protein fragments or antigens, is incubated with the test system.

Such a test system is also suitable for identifying binding agents according to the invention.

The invention furthermore relates to a diagnostic agent (synonym: array or assay) for implementing the test systems according to the invention and, where appropriate, to a pharmaceutically acceptable support.

Examples of pharmaceutically acceptable supports are glass, polystyrene, polypropylene, dextran, nylon, amylase, naturally or modified cellulose, polyacrylamides, agarose, aluminum hydroxide or magnitide. The support can also consist of plates containing 96 wells or more.

The diagnostic agent can be present in solution, be bound to a solid matrix and/or be treated with an adjuvant.

The diagnostic agent can also be administered to a patient in vivo as desired (e.g. capsule or tablet). A diagnostic agent according to the invention is therefore suitable for diagnosing diseases, specifically allergies, autoimmune diseases, in particular rheumatoid arthritis, multiple sclerosis or Crohn's disease, chronic inflammation, asthma, immunodeficiency diseases, AIDS, transplant rejection and cancer diseases as well as diabetes.

In particular for diagnosing autoimmune diseases, specifically alopecia greata, Bechterew's disease, antiphospholipid syndrome, Addison's disease, Behcet's disease, sprue celiac disease, chronic fatigue immune dysfunction syndrome (CFIDS), polyneuropathy, Churg-Strauss syndrome (granulomatosis), CREST syndrome (Raynaud's syndrome), cold agglutinin disease, cryoglobulinemia, fibromyalgia, fibromyositis, Basedow's disease, Guillain-Barré syndrome, idiopathic pulmonary fibrosis, idiopathic thrombocytopenia, IgA nephropathy, lichen planus, Ménière's disease, polyarteritis nodosa, polychondritis, polyglandular syndrome, polymyalgia rheumatica, primary agammaglobulinemia, biliary cirrhosis, psoriasis, Reiter's disease, sarcoidosis, Sjögren's disease, Takayasu arteritis, vasculitis, vitiligo and Wegener's granulomatosis.

The following examples serve to clarify the invention further without restricting it to these examples. Figures and sequences are also explained.

### EXAMPLES

### Example 1

### Isolation and Functional Analysis of Human Tregs

The T cells were isolated from PBMCs (peripheral blood mononuclear cells) which by a standard density gradient centrifugation from normal buffy coats or leukapherisates from healthy human donors.

### Example 1a

### CD4+ CD25+ Regulatory T Cells (CD25+ Tregs)

The leukapherisate from healthy voluntary donors which is prepared by the Mainz Transfusion Center and which on average contains 7-10×10⁹ leukocytes is used as the starting material.

In the first procedural step, the mononuclear cells are isolated by means of Ficoll gradient centrifugation and then washed intensively with PBS+1 mM EDTA.

The isolated leukocytes are then taken up in PBS+0.5% HAS (human serum albumin)+1 mM EDTA and incubated, at 4° C. for 15 min, with anti-CD25 Microbeads (2 μl of Microbeads/10⁷ leukocytes, Microbeads: Miltenyi GmbH, Bergisch-Gladbach, Federal Republic of Germany). After the incubation, the leukocytes are washed 2× with PBS+1 mM EDTA.

In order to isolate the CD25+ leukocytes, the cells are then loaded onto a separation column (LS Columns, Miltenyi) and separated in a permanent magnet (Miltenyi). The average yield of CD25+ leukocytes is 1.2-2% (purity >97%).

In order to deplete CD4-negative contaminations, the CD25+ leukocytes are then incubated, for 20 minutes in X-VIVO-15, with CD8, CD19 and CD14 Dynabeads (Dynal, Hamburg, FRG, 3 beads/cell) and mouse IgG1 anti-human CD45RA monoclonal antibodies (Coulter/Immunotech, Hamburg, FRG, 1 μg of Mab/10⁶ leukocytes). The bound CD8+, CD19+ and CD14+ contaminations can be removed directly using a permanent magnet (Dynal), while the CD45RA+ cells are removed in the permanent magnet using anti-mouse IgG Dynalbeads (Dynal). This depletion step is then repeated once again (purity of the CD4+ CD25+ leukocytes >95%).

### Example 1b

### α4β1+ and α4β7+ Subpopulations of Human Regulatory T Cells

Human CD25+ Tregs contain two functionally different subpopulations which differ in the expression of integrins. Approx. 20% of the Tregs express the α4β7 integrin while 80% express the α4β1 integrin. The following changes in the isolation protcol are required in order to isolate these subpopulations:

### In the first procedural step, the isolated leukocytes are incubated with mouse IgG anti-human CD25-FITC Mab (2 μl of Mab/10⁷ leukocytes, M-A251, BD Pharmingen, San Diego, USA) for 15 min at 4° C. and then washed intensively with PBS+1 mM EDTA.

The FITC-positive cells are isolated using anti-FITC Multisort Microbeads (Miltenyi). The method is carried out in analogy with the direct isolation of CD25+ leukocytes using CD25 Microbeads. The Microbeads are then removed from the surface of the leukocytes by means of enzymic digestion in accordance with the manufacturer's (Miltenyi) instructions.

The CD4-negative contaminations are depleted as previously described using CD8, CD19 and CD16 Dynabeads while CD4SRA+ cells are not depleted (purity of CD4+ CD25+ T cells >95%.

In the next procedural step, the α4β7+ subpopulation is isolated. For this, the CD4+ CD25+ T cells are incubated with a rat IgG anti-human β7 integrin-PE Mab (BD-PharMingen, 2 μg/10⁷ cells) for 15 min at 4° C. and then washed intensively with PBS+1 mM EDTA. The method for isolating the β7+T cells is carried out in analogy with the isolation of CD25+ T cells using anti-PE Microbeads (Miltenyi), resulting in the purity of the CD4+ CD25+α4β7+ cells being >90%. The negative fraction expresses the α4β1 integrin (purity of the CD4+ CD25+α4β1+ cells >80%).

### Example 2

### Characterizing Human CD4+ CD25+ Regulatory T Cells

CD25+ Tregs are characterized by their inhibitory effect on the activation of CD4+ and CD8+ T cells in vitro.

The functional characterization of CD25+ Tregs in vitro is analyzed in coculture assays using CD4+ T helper cells. For this, the T cells are stimulated either with allogenic, mature dendritic T cells or polyclonally with anti-CD3+ anti-CD28 Mabs.

### Example 3

### Multisort Positive Selection of CD4+ CD25+ and CD4⁺ CD25+β7+ T Cells

Tregs were isolated in several steps. CD4+ T cells were first of all isolated using the CD4-MACS Multisort kit (Miltenyi, Bergisch-Gladbach, Germany) and the CD4+ CD25+ T cells were isolated from them using anti-CD25-FITC (M-A251, BD PharMingen, San Diego, USA) and anti-FITC-Multisort Beads (Miltenyi, Bergisch-Gladbach, Germany). After that, B cells, macrophages and CD8+ T cells were depleted using CD19, CD14 and CD8 Dynabeads (Dynal, Hamburg, Germany). β7-PE and anti-PE Beads (Miltenyi, Bergisch-Gladbach, Germany) were used for isolating CD4+ CD25β+7+ Tregs (subpopulation of the CD4+ CD25+ Tregs). The purity of the isolated cells was checked by means of FACS analysis.

### Example 4

### Functional Analysis of Freshly Isolated Human CD4+ CD25+ T Cells

While CD25 is a typical surface molecule on Tregs, it is not only expressed in this cell type. For this reason, a functional check was carried out on the suppressive properties of the isolated cells prior to each analysis.

### Example 5

### Polyclonal Stimulation Using Anti-CD3 and Anti-CD28 Monoclonal Antibodies

A constant number of conventional CD4+ T cells (1×10⁵/well) can be activated polyclonally using anti-CD3 (1 μg/ml, OKT-3) and anti-CD28 (2 μg/ml, CD28.2) monoclonal antibodies in the presence of a varying number of CD4+ CD25+ T cells (ratio of 1:1 to 1:4). The T cell proliferation was measured after three days of culture and a subsequent 16-hour pulsed treatment with 3HTdR (37 kBq/well). The cells which had been tested in this way were used for the proteome analyses.

Total cell lysates from cultured cells for the 2DE After a cell lysis, the proteins were extracted from the cells using a method which was slightly modified from that used by Klose (Klose, J. and Kobalz, U., Two-dimensional electrophoresis of proteins: an updated protocol and implications for a functional analysis of the genome. Electrophoresis 16, 1034-1059 (1995) and Klose, J. Fractionated extraction of total tissue proteins from mouse and human for 2-D electrophoresis. Methods Mol Biol 112, 67-85 (1999)). The cells were lysed mechanically, using ultrasonication and glass beads, in a phosphate buffer which contained protease inhibitors directed against a large number of different proteases. The nucleic acids which interfered with the 2D gel electrophoresis were digested within 20 min at room temperature by adding the nuclease benzonase. The proteins were dissolved in a urea and thiourea-containing buffer to which DTT had been added. Servalytes 2-4 were added for the isoelectric focusing of the proteins.

### Example 6

### Protein Separation by Means of 2DE

The isoelectric focusing (IEF) of the proteins was performed in accordance with the method of Klose (Klose, J., Protein mapping by combined isoelectric focusing and electrophoresis of mouse tissues. A novel approach to testing for induced point mutations in mammals. Humangenetic, 26, 231-243 (1975)) using carrier ampholytes in circular polyacrylamide gels under reducing conditions. The separations were carried out in a pH range of from 2 to 11 with the length of the IEF gels being 40 cm. The proteins which had been separated by IEF were then separated by means of SDS-PAGE in 15% polyacrylamide gels. Before being loaded onto the gel for the SDS-PAGE, the IEF gel strands were washed twice with running buffer (0.3% (w/v) Tris base, 1.44% (w/v) glycine, 0.1% (w/v) SDS) in order to remove excess DTT. The gel strand was then laid, free of air bubbles, on the SDS gel and fixed using a 1% agarose solution (containing bromophenol blue). The entry of the proteins into the gel took place at 65 mA for 15 min, while the separation took place within approx. 5 h at 100 mA in the case of analytical gels which were 0.75 mm in thickness or at 75 and 200 mA in the case of preparative gels which were 1.0 mm and, respectively, 1.5 mm in thickness. The separation distance was 30 cm.

### Visualizing the Proteins

In order to achieve a sensitivity in protein detection which was as high as possible, the analytical gels were stained with silver using a modified method of Heukeshoven and Dernick (Heukeshoven, J. and Dernick, R. Improved silver staining procedure for fast staining in PhastSystem Development Unit. I. Staining of sodium dodecyl sulfate gels. Electrophoresis 9, 28-32 (1988)) which was modified as described by Klose and Kobalz (Klose, J. and Kobalz, U. Two-dimensional electrophoresis of proteins: an updated protocol and implications for a functional analysis of the genome Electrophoresis 16, 1034-1059 (1995)). Since this method makes use of added glutaraldehyde and formaldehyde in order to increase the sensitivity, it is scarcely possible to carry out any subsequent mass spectrometric identification of the proteins. For this reason, a modified variant of the staining described by Blum et al. (Blum, H., Beier, H. and Gross, H. J. Improved silver staining of plant proteins, RNA and DNA on polyacrylamide gels. Electrophoresis, 8, 93-99 (1987)), which is compatible with mass spectrometry, was employed as required. The colloidal Coomassie staining described by Neuhoff et al. (Neuhoff V., Arold N., Taube D. and Ehrhardt W., Improved staining of proteins in polyacrylamide gels including isoelectric focusing gels with clear background at nanogram sensitivity using Coomassie Brilliant Blue G-250 and R-250. Electrophoresis 9, 255-262. (1988)) using Coomassie Brilliant Blue G-250 was used for the protein in preparative 2DE gels which were analyzed by mass spectrometry. Alternatively, particularly in the case of proteins which could not be stained with the colloidal Coomassie stain, the proteins were stained with silver using a modified protocol without any addition of glutaraldehyde (Blum, H., Beier, H. and Gross, H. J. Improved silver staining of plant proteins, RNA and DNA in polyacrylamide gels. Electrophoresis, 8, 93-99 (1987)).

### Example 7

### Differential Proteome Analysis

In the case of silver-stained gels, the polyacrylamide gels were digitalized for the image analysis, after they had been dried, using a transmitted-light scanner. The relative protein intensities were analyzed quantitatively using special image analysis software which was suitable for these analyses (ProteomWeaver Vers. 2.0, Definiens, Germany).

The proteins which were found using the image analysis were excised manually from the gels. Using a washing robot, the gel pieces were washed in each case three times alternately with in each case 10 μl of digestion buffer (10 mm NH₄HCO₃) and, respectively, digestion buffer/acetonitrile 1:1 in order to remove the dye and buffer additives. In the case of silver-stained spots, the silver was, before the washing, oxidized, at room temperature and within approx. 1 min, by adding 15 μl of destaining solution (100 mM potassium hexacyanoferrate(III)/30 mM sodium thiosulfate, 1:1). The gel pieces were then dehydrated in a vacuum centrifuge and treated with in each case 2 μl of a trypsin solution (0.05 μg of trypsin/ml in digestion buffer). The proteolytic cleavage took place at 37° C. for at least 4 h or overnight. The resulting proteolysis products were extracted from the gel matrix at room temperature and within 30 min by adding 5 μl of 0.1% TFA.

### Example 8

### MALDI-TOF Mass Spectrometry

The peptide masses of proteolytically cleaved proteins were determined using a MALDI-TOF mass spectrometer of the Ultraflex type (Bruker Daltonik, Bremen, Germany) In this method, the analytic molecules (peptides) are cocrystallized in a UV-active matrix. For the matrix solution, a saturated solution of α-cyano-4-hydroxycinnamic acid in 50% acetonitrile/0.1% TFA 1:1 (solution A) was diluted with solution A in the ratio of 1:1. Prior to the measurements, the peptides were adsorbed, for enrichment, on C18 material in ZipTips™ (activated with 10 μl of 0.1% TFA) by drawing up the analyte solution several times, then washed once with 10 μl of 0.1% TFA and subsequently eluted onto the sample plate with 1.2 μl of matrix solution. The peptide mass fingerprint spectra (PMFs) of the samples which had dried on the sample plate were measured at the following settings: acquisition method: reflector, voltage polarity: positive, acceleration voltage: 25 kv, reflector voltage: 26.3 kV, lens voltage: 6.2 kv, reflector detector voltage: 1.72 kV and deflection voltage: 0 kV.

A proteinScape® database calibration algorithm (Bruker Daltonik) was used to calibrate the mass spectra automatically for the trypsin autoproteolysis products and for known peptides which occurred repeatedly in the spectra and were derived from contaminations such as keratin. The peptide mass spectra were analyzed with the aid of a nonredundant NCBI protein database using the ProteinScape® metasearch machine and the MASCOT and ProFound (version 2002.03.01) search algorithms.

### Analysis:

When the different T cell populations were compared, the quantity of the Charcot-Leyden crystal protein (galectin 10) was found to be increased in stimulated and unstimulated CD4+ CD25+ T cells as compared with the unstimulated CD4+ T cells. These results were found in four human donors who were independent of each other. In the case of two donors, an increase in the quantity of the Charcot-Leyden crystal protein was also found in a stimulated CD4+ CD25+β7+ T cell subpopulation (Treg) (FIG. 1, FIG. 2 and FIG. 3).

The Charcot-Leyden crystal protein was detected and identified in the gels in three isoforms having different molecular weights and isoelectric points. Isoform 1 (spot 68) had an apparent molecular weight of approx. 14 kDa and a pI of 6.7 while isoform 2 (spot 33) had an apparent molecular weight of approx. 13.5 kDa and a pI of 5.9 and isoform 3 (spot 34) had an apparent molecular weight of approx. 13 kDa and a pI of 5.9.

All the isoforms were identified as being Charcot-Leyden crystal protein (galectin 10) (SEQ ID No. 1 or SEQ ID No. 2).

The three isoforms exhibited coregulation in the T cell populations which were investigated.

Galectin 1 (SEQ ID No. 4) was likewise found to be present in higher concentration in the stimulated and unstimulated CD4+ CD25+ T cells as compared with the unstimulated CD4+ T cells.

These results were found in four independent human donors. In the case of two donors, a reduction in the quantity of the galectin 1 protein was found in a CD4+ CD25+β7+ T cell subpopulation (Treg) (FIG. 4). Comparative investigations on the same cell populations were also carried out in mice (inbred strain employed: BALB/c). The sequences of the corresponding galectin proteins are SEQ ID No. 3 and SEQ ID No. 5.

### Example 9

### Isolation and Stimulation of Human T Cell Populations

Conventional CD4+ CD25− T effector cells (in a subsequent text termed CD4+ T cells) and CD4+ CD25+ T cells (in a subsequent text termed CD25+ Treg cells) were isolated from buffy coats and leukapherisates from healthy human donors. CD25+ cells were isolated using CD25 Microbeads (Miltenyi). This resulted in CD25high cells. Contaminations with CD4− cells were then [lacuna] by depleting with CD14, CD8 and CD19 Dynabeads (Dynal). This purification step resulted in a population of CD4+ CD25high T cells which had a purity of >95%. (In some cases, CD25+CD45RA+ T cells were depleted using anti-CD45RA Mab (Pharmingen) in combination with anti-mouse IgG Dynabeads. This resulted in CD4+ CD25+CD45RO+ T cells (purity >96%). CD4+ CD25− T cells were isolated using CD4 Microbeads and then depleted of CD25+ T cell contaminations using CD25 Dynabeads (purity of the CD4+ CD25− T cells >98%). α4β7+ and α4β1+ Treg subsets were isolated for some of the analyses. The CD4+ CD25+ T cells were isolated using anti-CD25-FITC Mab in combination with anti-FITC Multisort beads (Miltenyi) and then further purified by depleting CD4− contaminations. The β7 integrin-positive subset of the Treg cells was isolated using anti-β7 integrin-PE Mab in combination with anti-PE Microbeads, with this resulting in two populations: CD4+ CD25+β7+ T cells (purity >95%, selected positively) and CD4+ CD25+β7− T cells (purity >90%, selected negatively). 1 μg of anti-CD3 (OKT-3)/ml and 2 μg of anti-CD28 (CD28.2, Pharmingen)/ml were used for the polyclonal activation of the T cells. A suboptimal stimulation of the cells with 0.5 μg of anti-CD3 (OKT-3)/ml and gamma radiation-inactivated PBMCs was used for proliferation assays of the cells. The cells were always cultured in serum-free X-VIVO-15 medium (Cambrex).

### Example 10

### Cloning, Recombinant Production and Purification of a His-Galectin 10 Fusion Protein

The galectin 10 gene was amplified from human leukocytes using Quick-Clone cDNA (BD Biosciences). The N terminal His tag galectin 10 construct (pET16b) was transfected into the Escherichia coli strain BL21(DE3) and expression was induced with 1 mM isopropyl-beta-D-thiogalactopyranoside (IPTG, Sigma). The cells produced the His galectin 10 fusion protein in the presence of 1M sorbitol and 2.5 mM betaine. The recombinant His-galectin 10 fusion protein was purified by means of Ni—NTA affinity chromatography (Qiagen). The identity of the purified protein was confirmed by means of MALDI mass spectrometry.

### Example 11

### Preparation of the siRNA and Nucleofection

Two sequences which were in each case 19 base pairs (bp) in length were selected from the galectin 10 sequence and synthesized, with a 2 bp overhang being synthesized in addition. The dsRNA which exhibited the greatest ability to suppress galectin 10 mRNA expression was selected:

galectin 10 sense: GGA GGA AUC AGA CAU UGU CdTdT;
galectin 10 antisense: GAC AAU GUC UGA UUC CUC CdTdT.

The siRNA was diluted in RNase-free water and stored at −80° C. The nucleofection was carried out in accordance with an Amaxa protocol which was optimized for T cells and using the primary human T cell Nucleofector™ kit (Amaxa). For this purpose, 3×10⁶ CD25+ T cells were suspended in the Nucleofector™ solution (Amaxa) and incubated with siRNA at concentrations of from ˜0.5 μM to 1 μM. Directly following the nucleofection, the cells were resuspended in warm X-VIVO-15 (Cambrex).

### Example 11

### Use of RT-PCR to Quantify Galectin mRNA in T Cells

Human galectin 10 mRNA was quantified in the following T cell populations: CD4+ cells which were unstimulated and stimulated polyclonally with anti-CD3/CD28 for 24 h, CD4+ CD25+β1+ cells which were unstimulated and stimulated polyclonally with anti-CD3/CD28 for 24 h, and CD4+ CD25+β7+ cells which were unstimulated and stimulated polyclonally with anti-CD3/CD28 for 24 h. The total cell RNA was isolated from 1×10⁶ cells using TRIZOL (Invitrogen, Karlsruhe, Germany). The corresponding cDNA was synthesized using RevertAid M-MulV reverse transcriptase in accordance with the manufacturer's instructions (MBI Fermentas, St. Leon-Rot, Germany). The RT-PCR was carried out using the following reaction mixture: 25 μl of reaction mixture containing 2.5 mm MgCl₂, 0.2 mM dNTP, 0.5 μM forward and reverse primers and 0.25 U of Biotherm DNA polymerase (GeneCraft, Germany).

The following PCR program was employed: 94° C. for 2 min, and in each case 35 cycles of 94° C. for 30 s, 55° C. for 30 s and 72° C. for 1 min.

The following primers, which extend over the intron/exon boundary of the sought-after cDNA were designed in order to avoid amplifying genomic DNA:

galectin-10.forward:
5′-TAC CCG TGC CAT ACA CAG AGG CTG-3′
galectin-10.reverse:
5′-CTT ATC TGG CAG CAC TGA GAT GCT C-3′
hβ-actin. forward:
5′-GAG CGG GAA ATC GTG CGT GAC-3′
hβ-actin. reverse:
5′-GAA GGT AGT TTC GTG GAT GGC-3′
18S rRNA. forward:
5′-TCG ATG CTC TTA GCT GAG TGT CC-3′
18S rRNA. reverse:
5′-TGA TCG TCT TCG AAC CTC CG-3′
EF1-α.forward:
5′-GAT TAC AGG GAC ATC TCA GGC TG-3′
EF1-α.reverse:
5′-TAT CTC TTC TGG CTG TAG GGT GG-3′
FoxP3.forward:
5′-CTA CGC CAC GCT CAT CCG CTG G-3′
FoxP3.reverse:
5′-GTA GGG TTG GAA CAC CTG CTG GG-3′

The real-time analysis of galectin 10 mRNA was carried out using the iCycler (Bio-Rad, Munich, Germany) and employing IQ SYBRO Green Supermix (Bio-Rad). The relative expression levels of galectin 10 mRNA were calculated after the intensities had been normalized to the expression of 18S rRNA.

### Example 12

### Producing a Monoclonal Anti-Galectin 10 Antiserum

Rabbits were immunized with the recombinant galectin 10 using 50 μg of protein in solution. This solution was emulsified with an equal volume of complete Freund's adjuvant (CFA) and injected intravenously at several sites along the back of the rabbit. Additional booster injections of galectin 10 in CFA were given three times within a period of three weeks. The production of antibodies was monitored by means of ELISA and Western blot analyses. After three final bleedings, the IgG was isolated from the antiserum using a method developed by Harboe and Ingild (Harboe N and Ingild A. “Immunization, isolation of immunoglobulins, estimation of antibody titre.” Scand J Immunol Suppl. 1:161 (1973)).

### Example 13

### Western Blot Analysis

For the Western blot analyses using 1D PAGE, the cells were lysed in SDS buffer and the protein concentration was analyzed using a TLC protein assay (Bio Rad, Munich, Germany). Serum albumin was employed as the standard. The proteins were separated, at the rate of 5-10 μg/well, in 16% Tricin SDS polyacrylamide gels and then transferred to membranes. Nonspecific binding sites were saturated with Roti-Block (Roth, Karlsruhe, Germany). For the immunodetection, the membranes were in each case incubated for 1 hour firstly with the anti-galectin 10 antibody and then with a horseradish peroxidase-conjugated secondary anti-rabbit antibody. The peroxidase activity was visualized by means of a color reaction using 3,3′-diaminobenzidine (DAB, DakoCytomation, Copenhagen, Denmark).

For Western blot analyses using 2D PAGE, 60 μg of the soluble proteins from the total cell lysates were separated in a 2D gel and the proteins were transferred to a nitrocellulose membrane. After nonspecific binding sites had been saturated with Roti-Block overnight, the membranes were incubated with 2 μg of anti-galectin 10 antibody for 1 hour and then with an alkaline phosphatase-conjugated anti-rabbit antibody (Sigma, Taufkirchen, Germany) for 1 hour. The membranes were stained with NBT/BCIP. The signals which were detected in the 2D Western blot were lined up with the silver-stained proteins in the 2D gels.

### Example 14

### Immunocytochemistry

Cytocentrifugational preparations of freshly isolated CD4+ or CD25+ T cells were dried in air and stored at −20° C. until stained. For the staining, the sample carriers were briefly thawed and then fixed in 4% paraformaldehyde for 15 min at room temperature. The cells were washed with PBS and incubated with 50 mM NH₄Cl in PBS for 10 minutes. After that, the cells were permeabilized with 0.2% Triton X-100 on ice and within 5 minutes. After washing with PBS, the cytospins were incubated with a peroxidase-blocking solution (DakoCytomation, Copenhagen, Denmark) for 5 minutes in order to neutralize endogenous peroxidase activity. Nonspecific binding sites were then saturated with 20 μg of goat serum (normal goat serum; Santa Cruz Biotechnology, Santa Cruz, USA)/ml. The immunodetection was effected, overnight at 4° C., by adding 10 μg of anti-galectin 10 antibody/ml or using the preimmune serum as control. The cytospins were incubated with a horseradish peroxidase-conjugated anti-rabbit goat antibody (554021, BD Biosciences Pharmingen). After that, the cells were washed intensively with PBS and the peroxidase activity was visualized by means of a color reaction using 3,3′-diaminobenzidine (DakoCytomation, Copenhagen, Denmark).

### Example 15

### Comparative Proteome Study of Human CD25+ Tregs Versus Conventional CD4+ T Cells

Naturally occurring CD25+ Tregs are characterized by the unique property of suppressing the activation of conventional CD4+ T cells. However, until now, little has been known about the proteins which are involved in this cell-contact-dependent process. A differential proteome analysis of resting and activated conventional CD4+ T cells as compared with resting and activated CD25+ Treg cells was carried out in order to identify the proteins which are involved in the function of the CD25+ Treg cells. For this purpose, up to 10⁸ CD25+Treg and CD4+ T cells were isolated, at a very high degree of purity, from buffy coats or leukapheresates. Prior to the proteome analysis, the T cell preparations were characterized with regard to their functionality. The resting cells were analyzed by 2D PAGE immediately after having been isolated while the activated cells were activated polyclonally for 48 hours.

The 2D gels which were used for the proteome study covered a pI range of from 4 to 10 and a molecular weight range of from 6 to 150 kDa.

When comparing the gels, approx 1600 protein spots were detected, and matched, in all the gels. The gels for a sample were prepared as a triplicate determination, in connection with which the protein spot patterns were not only very similar to each other within a sample but also when comparing the different T cell populations and also the individual human donors who were investigated. The greatest proportion (>90%) of all the depictable protein spots displayed a high degree of reproducibility both with regard to the relative position in the 2D gel and with regard to the spot intensity. With an increase in intensity by a factor of from 10 to 40, the galectin 10 isoforms 1 to 3 exhibit the greatest differences in the comparison.

The expression of galectin 10 has thus far only been reported in granulocytes (Golightly, L. M., Thomas, L. L., Dvorak, A. M. and Ackerman, S. J. “Charcot-Leyden crystal protein in the degranulation and recovery of activated basophils” J. Leukoc. Biol. 51, 386-392 (1992); Dvorak, A. M., Letourneau, L., Weller, P. F. and Ackerman, S. J. “Ultrastructural localization of Charcot-Leyden crystal protein (lysophospholipase) to intracytoplasmic crystals in tumor cells of primary solid and papillary epithelial neoplasm of the pancreas” Lab. Invest. 62, 608-615 (1990); Dvorak, A. M. and Ackerman, S. J. “Ultrastructural localization of the Charcot-Leyden crystal protein (lysophospholipase) to granules and intragranular crystals in mature human basophils” Lab. Invest. 60, 557-567 (1989)).

### Example 16

### Detecting the Expression of Galectin 10 mRNA in Human CD25+ Treg Subsets

The results of the proteome analysis of human T cells showed that galectin 10 protein is produced to the greatest extent by CD25+ Tregs. Conventional RT-PCR and real-time PCR were used to analyze these results further at the mRNA level. In contrast to the protein data from the proteome study, no galectin 10 mRNA was detected in conventional CD4+ T cells even after 30 RT-PCR cycles (FIG. 6A). However, a very strong signal for galectin 10 mRNA was detected under the same conditions in freshly isolated CD25+ Tregs.

In further analyses, the content of galectin 10 mRNA in conventional CD4+ T cells and CD25+ Treg cells was investigated by means of quantitative real-time PCR. While freshly isolated CD4+ T cells express very low quantities of galectin 10 mRNA, freshly isolated CD25+Treg cells express large quantities of galectin 10 mRNA. The mRNA levels decrease in both cell populations following polyclonal activation. No galectin 10 mRNA can any longer be detected in CD4+ T cells 48 hours after the activation.

### Example 17

### Western Blot Analysis of Galectin 10 in Cell Lysates from Resting and Activated Human CD4+ T Cells and CD25+ Treg Cells

In order to verify the data from the proteome analysis using Western blot analyses, recombinant galectin 10 was prepared and a polyclonal antiserum was generated from it. The IgG fraction of the antiserum which was prepared was used for detecting galectin 10 in lysates of resting and activated conventional human CD4+ T cells and CD25+ Treg cells. The proteins in the lysates were separated beforehand by means of one dimensional or two dimensional gel electrophoresis. FIG. 7 shows that, while galectin 10 is scarcely detectable in lysates of conventional CD4+ T cells, strong staining can be detected in the lysates from CD25+ Treg cells. The recombinant galectin 10 was used as a positive control in this experiment. The Western blot which is shown in FIG. 7 depicts a representative result from seven independent experiments carried out on cells from independent healthy donors. In addition, this result shows that the antibody prepared against the recombinantly prepared galectin 10 also recognizes the natural galectin 10 protein. Three different isoforms of the galectin 10 protein were detected in the proteome analyses and identified by mass spectrometry. Western blot analyses which were carried out after the proteins from the lysate of human CD25+ Treg cells had been separated showed that the antibody stains all three isoforms of the protein. In addition, very weak signals for two (four) further iso forms of the protein were also obtained. It was possible to line up these signals with the silver-stained 2D gels (FIG. 3B).

### Example 18

### Staining Conventional CD4+ T Cells and CD25+ Treg Cells with the Rabbit Anti-Galectin 10 IgG

The fact that the protein galectin 10 is almost exclusively detected in CD25+ regulatory T cells demonstrates its potential as a marker for these cells, in order to differentiate between conventional CD4+ T cells and CD25+ regulatory cells. In order to show this, cytospin preparations of the two T cell populations which had been isolated from the same donor were stained. FIG. 8 shows that only a small proportion, of less than 1%, of the freshly isolated conventional CD4+ T cells was stained. This small proportion is probably the result of a slight contamination with CD25+ T cells. In the population of CD25+ regulatory T cells, 20-30% exhibited strong staining while the other cells did not become stained. This result demonstrates that the CD4+ CD25+ T cells which were isolated from peripheral blood using antibody-coupled magnetic beads are not a homogeneous cell population but are, instead, composed of activated conventional CD4+ CD25+ T cells and CD25+ regulatory T cells. The staining of the cells also provides information with regard to the subcellular location of galectin 10 in regulatory T cells. In the few positively stained cells in the population of conventional CD4⁺ T cells, galectin 10 was distributed uniformly in the cell whereas a concentration of galectin 10 at the plasma membrane could be detected in the CD25+ regulatory T cells. Based on the different staining behavior (presence of galectin 10), it is possible to distinguish between conventional CD4+ T cells and CD25+ regulatory T cells by staining with antibodies or binding agents directed against galectin 10.

### Example 19

### Functional Properties of Galectin 10 in Human CD25+ Regulatory T Cells

Inhibiting galectin 10 expression with siRNA abolishes the anergic state of these cells and reduces their suppressive ability. A suitable siRNA was used to inhibit galectin 10 expression in order to characterize the function of this protein within the CD25+ regulatory T cells in more detail. For this purpose, CD25+ regulatory T cells were transfected with galectin 10 siRNA by means of nucleofection and the rate at which galectin 10 mRNA was expressed was quantified. The content of galectin 10 mRNA was most strongly reduced at 48 hours after the transfection. FIG. 9 shows the expression of galectin 10 mRNA as reduced by galectin 10 siRNA. At 0.5 μM galectin 10 siRNA, the galectin 10 mRNA is reduced down to 27% of the content in the control, while the galectin 10 mRNA is reduced down to 11% of the content in the control at 1.0 μM galectin 10 siRNA (SC=scrambled control: 0.5 μM and 1.0 μM). After having reached a maximum suppression of galectin 10 mRNA, the SiRNA-transfected CD25+ T cells were activated polyclonally for 48 hours with anti-CD3 and anti-CD28 monoclonal antibodies. The proliferation of these cells was monitored over a period of a further 96 hours by the uptake of radioactively labeled thymidine. The proliferation of the siRNA-transfected cells was markedly higher than in the case of the corresponding control cells (SC scrambled controls: 0.5 μM and 1 μM) and reached a proliferation which corresponds to that of conventional. CD4+ T cells (FIG. 9). There was no change in the proliferation of conventional CD4+ T cells which were transfected analogously with the galectin 10 siRNA. These results demonstrate that galectin 10 has a crucial function in maintaining the anergic state of the CD25+ regulatory T cells.

In order to investigate the influence of galectin 10 on the suppressive properties of the CD25+ regulatory T cells, the latter were cocultured, after having been transfected with galectin 10 siRNA, with conventional CD4+ T cells. The proliferation of the conventional CD4+ T cells was not altered in this connection. This means that the presence of galectin 10 protein in the CD25+ regulatory T cells is essential for the suppressive properties of the cells.

### Example 20

### Cryopreservation of T Cells

In order to cryopreserve T cells, a cell pellet was taken out in 50 μl of Tissue-Tek (Miles Diagnostic, Elkhart USA) and suspended by means of careful stirring. This cell suspension was deep-frozen dropwise in liquid nitrogen. A frozen drop was transferred to a cryoplastic form, which was filled with Tissue-Tek and once again frozen in liquid nitrogen.

The sections were prepared with a thickness of 3 μm and subsequently dried overnight at room temperature. Cryosections were stained with antibodies in analogy with the cytospin preparations.

### Explanation of the Sequences:

SEQ ID No. 1, Human Charcot-Leyden crystal protein
(galectin 10):
1HDK
A Chain A, Charcot-Leyden Crystal Protein - Pcmbs
Complex

| | |
|---|---|
| ACCESSION | 1HDK; gi\|17942629 |
| Organism: | Homo sapiens |

SLLPVPYTEAASLSTGSTVTIKGRPLVCFLNEPYLQVDFHTEMKEESDIV
FHFQVCFCRRVVMNSREYGAWKQQVESKNMPFQDGQEFELSISVLPDKYQ
VMVNGQSSYTFDHRIKPEAVKMVQVWRDISLTKFNVSYLKR
SEQ ID No. 2:
Q05314
Eosinophil lysophospholipase (Charcot-Leyden
crystal protein). (Lysolecithin acylhydrolase)
(CLC) (galectin-10).

| | |
|---|---|
| ACCESSION | Q05315; gi\|547870 |
| Organism: | Homo sapiens |

MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDISLTKFNVSYLKR
SEQ ID No. 3, mouse Charcot-Leyden crystal
protein homolog:
P97400
Eosinophil lysophospholipase (Charcot-Leyden
crystal protein homolog). (Lysolecithin acylhy-
drolase) (CLC) (galectin 10).

| | |
|---|---|
| ACCESSION | P97400; gi\|2829838 |
| Organism: | Mus musculus |
| or | |
| AAB41694 | |

Charcot-Leyden crystal protein ortholog

| | |
|---|---|
| ACCESSION: | AAB41694; gi\|1813526 |
| Organism: | Mus musculus |

EPYLQVDFHTEMKEDSDIAFHSRVYFGHWVVMNSRVNGAWQYEVTCHNMP
FQDGKPFNLSISVPPDKY
SEQ ID No. 4, human galectin 1:
NP_002296
beta-galactosidase binding lectin precursor;
lectin,galactose-binding, soluble, 1; galectin

| | |
|---|---|
| Organism: | Homo sapiens |
| ACCESSION | NP002296; gi\|450981 |

MACGLVASNLNLKPGECLRVRGEVAPDAKSFVLNLGKDSNNLCLHFNPRF
NAHGDANTIVCNSKDGGAWGTEQREAVFPFQPGSVAEVCITFDQANLTVK
LPDGYEFKFPNRLNLEAINYMAADGDFKIKCVAFD
SEQ ID No. 5, mouse galectin 1:
P16045
Galectin-1 (beta-galactoside-binding lectin L-14-
I) (lactose-binding lectin 1) (S-Lac lectin 1)
(galaptin) (14 kDa lectin)

| | |
|---|---|
| ACCESSION: | P16045, gi\|126172 |

MACGLVASNLNLKPGECLKVRGEVASDAKSFVLNLGKDSNNLCLHFNPRF
NAHGDANTIVCNTKEDGTWGTEHREPAFPFQPGSITEVCITFDQADLTIK
LPDGHEFKFPNRLNMEAINYMAADGDFKIKCVAFE
SEQ ID No. 6, nucleic acid encoding an amino acid
sequence as depicted in SEQ ID No. 1 or SEQ ID No.
2 (galectin 10):
CAATTCAGAAGAGCCACCCAGAAGGAGACAACAATGTCCCTGCTACCCGT
GCCATACACAGAGGCTGCCTCTTTGTCTACTGGTTCTACTGTGACAATCA
AAGGGCGACCACTTGTCTGTTTCTTGAATGAACCATATCTGCAGGTGGAT
TTCCACACTGAGATGAAGGAGGAATCAGACATTGTCTTCCATTTCCAAGT
GTGCTTTGGTCGTCGTGTGGTCATGAACAGCCGTGACTATGGGGCCTGGA
AGCAGCAGGTGGAATCCAAGAACATGCCCTTTCAGGATGGCCAAGAATTT
GAACTGAGCATCTCAGTGCTGCCAGATAAGTACCAGGTAATGGTCAATGG
CCAATCCTCTTACACCTTTGACCATAGAATCAAGCCTGAGGCTGTGAAGA
TGGTGCAAGTGTGGAGAGATATCTCCCTGACCAAATTTAATGTCAGCTAT
TTAAAGAGATAACCAGACTTCATGTTGCCAAGGAATCCCTGTCTCTACGT
GAACTTGGGATTCCAAAGCCAGCTAACAGCATGATCTTTTCTCACTTCAA
TCCTTACTCCTGCTCATTAAAACTTAATCAAACTTCAAAAAAAAAAAA
SEQ ID No. 7, nucleic acid encoding an amino acid
sequence as depicted in SEQ ID No. 4 (galectin 1):
ATCTCTCTCGGGTGGAGTCCTTCTGACAGCTGGTGCGCCTGCCCGGGAAC
ATCCTCCTGGACTCAATCATGGCTTGTGGTCTGGTCGCCAGCAACCTGAA
TCTCAAACCTGGAGAGTGCCTTCGAGTGCGAGGCGAGGTGGCTCCTGACG
CTAAGAGCTTCGTGCTGAACCTGGGCAAAGACAGCAACAACCTGTGCCTG
CACTTCAACCCTCGCTTCAACGCCCACGGCGACGCCAACACCATCGTGTG
CAACAGCAAGGACGGCGGGGCCTGGGGGACCGAGCAGCGGGAGGCTGTCT
TTCCCTTCCAGCCTGGAAGTGTTGCAGAGGTGTGCATCACCTTCGACCAG
GCCAACCTGACCGTCAAGCTGCCAGATGGATACGAATTCAAGTTCCCCAA
CCGCCTCAACCTGGAGGCCATCAACTACATGGCAGCTGACGGTGACTTCA
AGATCAAATGTGTGCCCTTTGACTGAAATCAGCCAGCCCATGGCCCCCAA
TAAAGGCAGCTGCCTCTGCTCCCCTG
SEQ ID No. 8
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVOVWRDISLTKFNVSYLK
SEQ ID No. 9
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDISLTKFNVSYL
SEQ ID No. 10
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKNVQVWRDISLTKFNVSY
SEQ ID No. 11
MSLLPVPYTEAASLSTGSTVTIKGRPLVCFLNEPYLQVDFHTEMKEESDI
VFHFQVCFGRRVVNNSREYGAWKQQVESKNMPFQDGQEFELSISVLPDKY
QVMVNGQSSYTFDNRIKPEAVKMVQVWRDISLTKFNVS
SEQ ID No. 12
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDISLTKFNV
SEQ ID No. 13
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDISLTKFN
SEQ ID No. 14
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDISLTKF
SEQ ID No. 15
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDISLTK
SEQ ID No. 16
MSLLPVPYTEAASLSTGSTVTIKGRPLVCFLNEPYLQVDFHTEMKEESDI
VFHFQVCFGRRVVNNSREYGAWKQQVESKNMPFQDGQEFELSISVLPDKY
QVMVNGQSSYTFDHRIKPEAVKMVQVWRDISLT
SEQ ID No. 17
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDISL
SEQ ID No. 18
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVNVNGQSSYTPDHRIKPEAVKMVQVWRDIS
SEQ ID No. 19
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRDI
SEQ ID No. 20
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWRD
SEQ ID No. 21
NSLLPVPYTEAASLSTGSTVTIKGRPLVCFLNEFYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWR
SEQ ID No. 22
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVWR
SEQ ID No. 23
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQVW
SEQ ID No. 24
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQV
SEQ ID No. 25
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMVQ
SEQ ID No. 26
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKMV
SEQ ID No. 27
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVKM
SEQ ID No. 28
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAVK
SEQ ID No. 29
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEAV
SEQ ID No. 30
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPEA
SEQ ID No. 32
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKPE
SEQ ID No. 33
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRIKP
SEQ ID No. 34
MSLLPVPYTEAASLSTGSTVTIKGRPLVCPLNEPYLQVDFHTEMKEESDI
VFHFQVCFGRRVVMNSREYGAWKQQVESKNMPFQDGQEFELSISVLPDKY
QVMVNGQSSYTFDHRIK
SEQ ID No. 35
MSLLPVPYTEAASLSTGSTVTIKGRPLVCFLNEPYLQVDFHTEMKEESDI
VFHFQVCFGRRVVMNSREYGAWKQQVESKNMPFQDGQEFELSISVLPDKY
QVMVNGQSSYTFDHRI
SEQ ID No. 36
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRI
SEQ ID No. 37
Ac-SLLPVPYTEAASLSTGSTVTIKGRPLVCFLNEPYLQVDFHTENKEES
DIVFHFQVCFGRRVVMNSREYGAWKQQVESKNMPFQDGQEFELSISVLPD
KYQVMVNGQSSYTFDHRIKPEAVKMVQVWRDISLTKFNVSYLK
SEQ ID No. 38
Ac-SLLPVPYTEAASLSTGSTVTIKGRPLVCFLNEPYLQVDFHTEMKEES
DIVFHFQVCFGRRVVMNSREYGAWKQQVESKNMPFQDGQEEELSISVLPD
KYQVNVNGQSSYTFDKRIKPEAVKMVQVWRDISLTKFNVSYL
SEQ ID No. 39
Ac-SLLPVPYTEAASLSTGSTVTIKGRPLVCFLNEPYLQVDFHTEMKEES
DIVFHFQVCEGRRVVMNSREYGAWKQQVESKNMPFQDGQEFELSISVLPD
KYQVMVNGQSSYTFDHRIKPEAVKMVQVWRDISLTKFNVSY
SEQ ID No. 40
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWRDISLTKFNVS
SEQ ID No. 41
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWRDISLTKFNV
SEQ ID No. 42
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWRDISLTKFN
SEQ ID No. 43
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWRDISLTKF
SEQ ID No. 44
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWRDISLTK
SEQ ID No. 45
Ac-SLLPVPYTEAASLSTGSTVTIKGRPLVCFLNEPYLQVDFHTEMEEES
DIVFHFQVCFGRRVVMNSREYGAWKQQVESKNMPFQDGQEFELSISVLPD
KYQVMVNGQSSYTFDHRIKPEAVKMVQVWRDISLT
SEQ ID No. 46
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWRDISL
SEQ ID No. 47
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWRDIS
SEQ ID No. 48
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWRDI
SEQ ID No. 49
Ac-SLLPVPYTEAASLSTGSTVTIKGRPLVCFLNEPYLQVDFHTEMKLES
DIVFHFQVCFGRRVVMNSREYGAWKQQVESKNMPFQDGQEFELSISVLPD
KYQVMVNGQSSYTFDHRIKPEAVKMVQVWRD
SEQ ID No. 50
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWR
SEQ ID No. 51
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVWR
SEQ ID No. 52
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQVW
SEQ ID No. 53
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMVQV
SEQ ID No. 54
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVNVNGQSSYTFDHRIKPEAVKMVQ
SEQ ID No. 55
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKMV
SEQ ID No. 56
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVKM
SEQ ID. No. 57
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAVK
SEQ ID No. 58
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEAV
SEQ ID No. 59
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPEA
SEQ ID No. 60
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKPE
SEQ ID No. 61
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIKP
SEQ ID No. 62
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRIK
SEQ ID No. 63
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHRI
SEQ ID No. 64
Ac-SLLPVPYTEAASLSTGSTVTIKGRPLVCFLNEPYLQVDFHTEMKEES
DIVFHFQVCFGRRVVMNSREYGAWKQQVESKNMPFQDGQEFELSISVLPD
KYQVMVNGQSSYTFDHR

### EXPLANATION OF THE FIGURES

FIG. 1

Change in the concentration of Charcot-Leyden crystal protein isoform 1 (spot 68) when comparing human regulatory T cells (CD4+ CD25+ and CD4+ CD25+β7+) with conventional T cells (CD4+) following polyclonal stimulation with anti-CD3 and anti-CD28 antibodies. The arrows show the differential protein spots.

FIG. 2

Change in the concentrations of Charcot-Leyden crystal protein isoform 1 (spot 33) and isoform 3 (spot 34) when comparing human regulatory T cells (CD4+ CD25+ and CD4+ CD25+β7+) with conventional T cells (CD4+) following polyclonal stimulation with anti-CD3 and anti-CD28 antibodies. The arrows show the differential protein spots.

FIG. 3

Change in the concentration of Charcot-Leyden crystal protein isoform 1 (spot 68) when comparing stimulated and unstimulated human regulatory T cells (CD4+ CD25+ and CD4+ CD25+β7+) and conventional T cells (CD4+). The arrows show the differential protein spots.

FIG. 4

Change in the concentration of galectin 1 when comparing human regulatory T cells (CD4+ CD25+ and CD4+ CD25+β7+) and conventional T cells (CD4+) following polyclonal stimulation with anti-CD3 and anti-CD28 antibodies. The arrows show the differential protein spots.

FIG. 5:

Quantification of galectin 10 (spot 68) spot intensities following separation of the total lysates of resting and 48 h-activated conventional T cells and Tregs. The spot intensities were determined using the Proteomweaver image analysis software and in each case normalized to the spot intensity in activated conventional CD4+ T cells (relative intensity=1). Intensities which were approx. 40-fold higher than in conventional T cells were determined in both resting and activated CD4+ CD25+ Tregs.

FIG. 6:

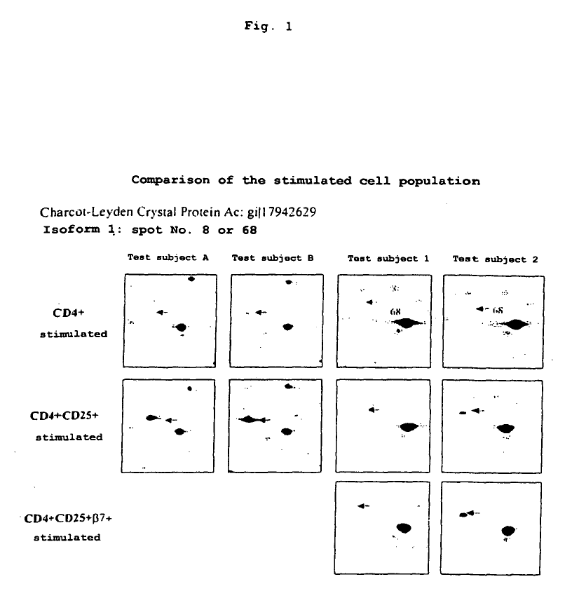

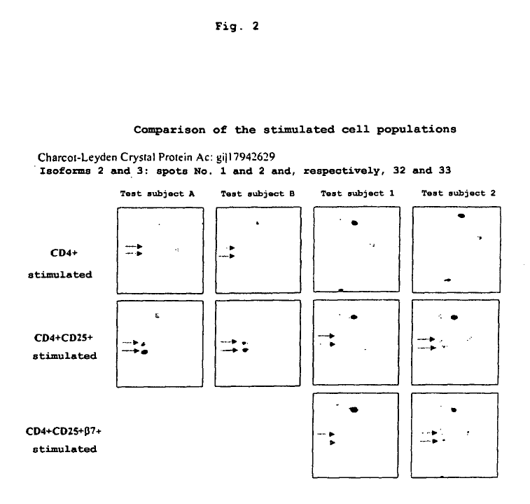

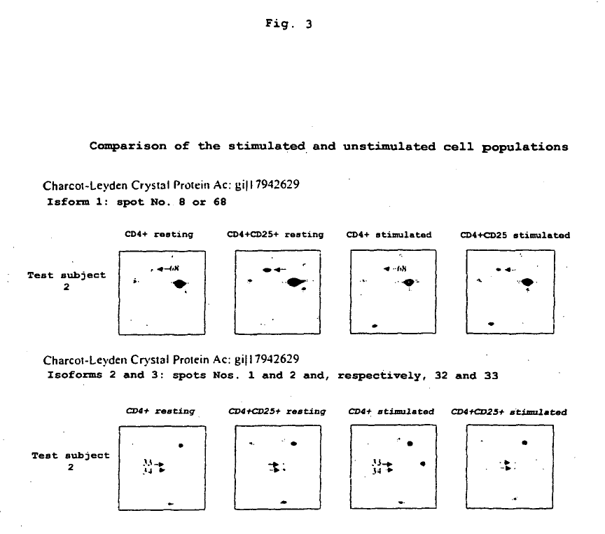

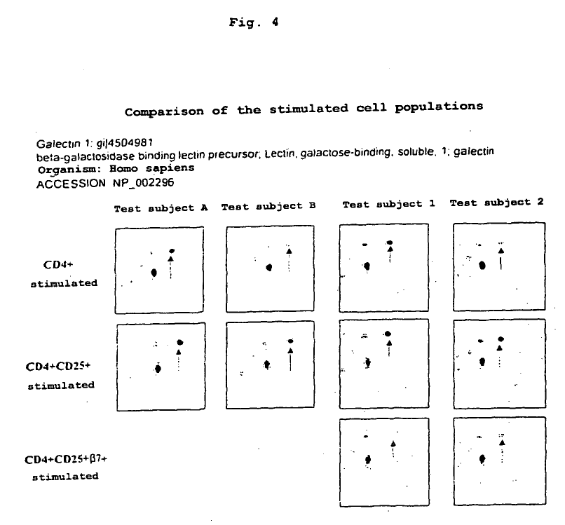

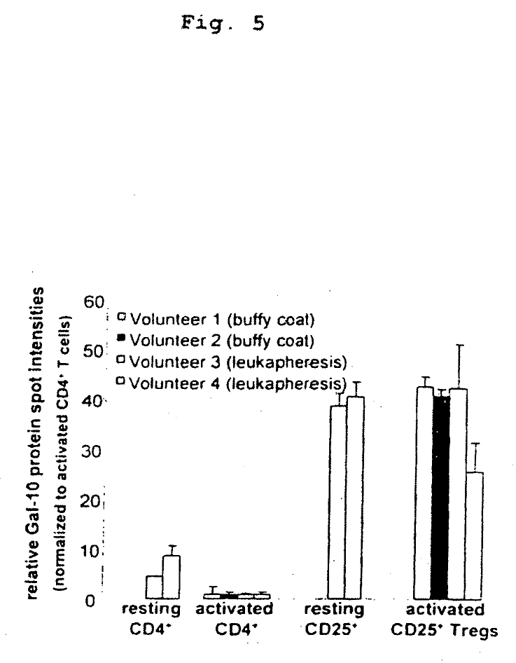

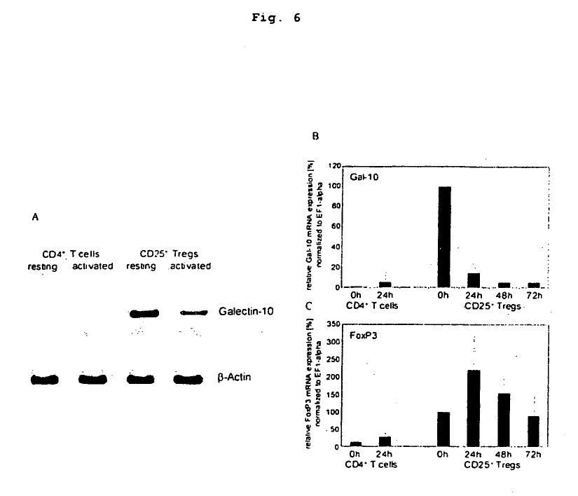

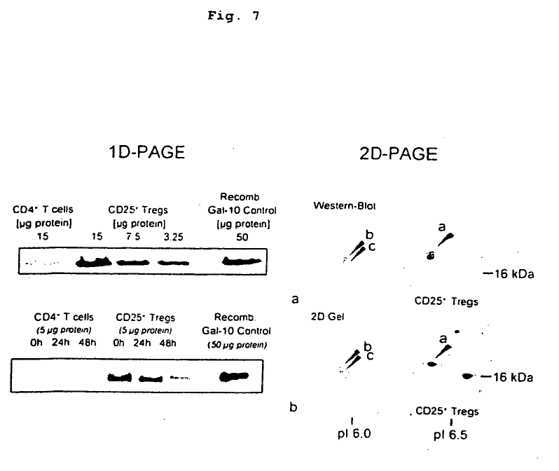

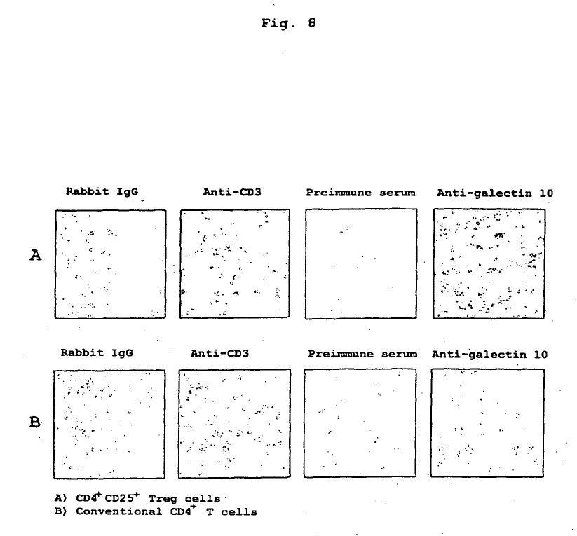

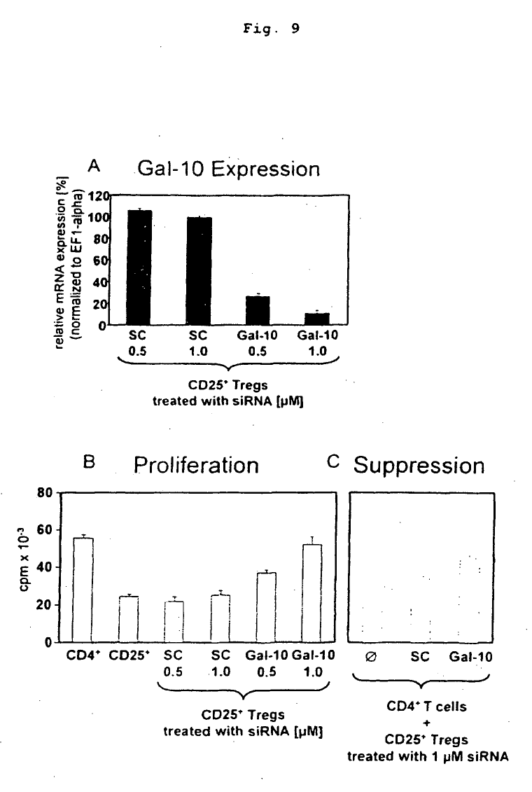

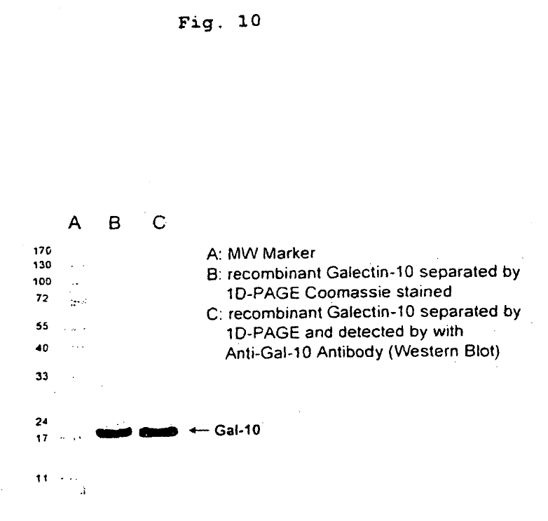

Expression of Galectin 10 mRNA in CD25+ Tregs

A: RT-PCR analysis of the galectin 10 mRNA and β-actin mRNA from freshly isolated and activated conventional CD4+ T cells and CD25+ Tregs.

B: Quantification of the relative galectin 10 mRNA in CD4+ T cells and CD25+ Tregs. cDNA samples were analyzed by means of quantitative real-time PCR using specific primers for galectin 10 or EF1-α. The relative content of galectin 10 mRNA in each sample was normalized to the content of EF1-α mRNA.

C: Quantification of the relative content of FoxP3 mRNA in CD4+ T cells and CD25+ Tregs. cDNA samples were analyzed by means of quantitative real-time PCR using specific primers for FoxP3 or EF1-α. The relative content of the FoxP3 mRNA in each sample was normalized to the content of EF1-α mRNA.

(activated=activated for 24 hours with anti-CD3 Mab [0.5 μg/ml] and anti-CD28 Mab [1 μg/ml]).

FIG. 7: Western blot analysis of the production of galectin 10 in CD25+ Tregs and conventional CD4+ T cells

A 1D PAGE: Freshly isolated (T=0 hours) and activated T cells (1 μg of monoclonal anti-CD3 antibody/ml and 2 μg of monoclonal anti-CD28 antibody/ml for the specified period) were lysed and 5 μg of the total protein extract were separated by means of 1D PAGE. The proteins, which were immobilized on a membrane, were incubated with 0.5 μg of anti-galectin 10 IgG. The blot was visualized by means of chemiluminescence using a horseradish peroxidase-conjugated anti-rabbit antibody and the ECL substrate.

B 2D PAGE: Western blot analysis following 2D PAGE of the galectin 10 isoforms. The total cell lysate from CD25+ Tregs which were isolated and activated for 48 hours with 1 μg of monoclonal anti-CD3 antibody/ml and 2 μg of monoclonal anti-CD28 antibody/ml was separated in a 2D gel. The immunoblotting was carried out in analogy with that performed after one-dimensional separation except that an alkaline phosphatase-conjugated secondary anti-rabbit antibody, and BCIP/NBT as substrate, were used for visualizing the isoforms. The signals of the 2D gel Western blot were lined up with a silver-stained 2D gel of the same T cells. All three of the proteins which had been previously identified as being galectin 10 were brought into line with the Western blot signals.

FIG. 8: Staining conventional CD4+ T cells and CD25+ Tregs with polyclonal anti-galectin 10 antibody

Cryosection preparations of activated conventional CD4+ T cells and CD25+ Treg cells. The cells which had been prepared in this way were stained with anti-CD3 antibodies in a control assay. This surface protein is expressed both on conventional CD4+ T cells and on Treg cells. The two cell populations were stained positively in the cryosections. The secondary antibody anti-rabbit IgG was used as a negative control. Galectin 10 was stained with the anti-galectin 10 antiserum. In this connection, it was clearly demonstrated that galectin 10 can only be detected in Treg cells. The conventional T cells did not show any positive staining. The preimmune serum served as negative control.

FIG. 9: galectin 10 gene knock-out breaks the anergy of human CD25+ Tregs

A Galectin 10 expression: freshly isolated CD25+ Tregs were transfected with 0.5 μM or 1 μM siRNA directed against galectin 10 or with 1 μM control SiRNA (scrambled control: SC). 24 hours after transfection, the cells were lysed and the RNA was isolated and used for real-time PCR analyses. The quantity of galectin 10 mRNA was quantified and normalized to the quantity of the mRNA of the housekeeping gene EF1-α (NF=nucleofected without siRNA).

B Proliferation: 48 hours after the transfection, the T cells were stimulated with monoclonal anti-CD3 and anti-CD28 antibodies (1 μg/ml+2 μg/ml). The proliferation of the T cells was measured after a further 4 days by adding 37 kBq/well of 3H-Tdr for a further 16 hours.

C Suppression: the suppressive properties of the Tregs on conventional T cells, after transfecting the Tregs with galectin 10 siRNA, were determined by measuring the proliferation of the conventional T cells. For this, the two cell types were cultured in coculture. The proliferation of the Treg population had been inhibited beforehand by radioactive irradiation. The coculture experiments clearly showed that the suppressive properties of the Tregs declined after galectin 10 transcription, and consequently production of the protein, have been inhibited with siRNA.

FIG. 10: Monitoring the purity of the recombinantly prepared human galectin 10 and the selectivity of the polyclonal anti-galectin 10 antiserum which was prepared.

After the His tag in His-galectin 10 had been proteolytically eliminated with factor Xa, the protease (factor Xa) was removed using a benzamidine column. The moiety which had been cleaved off was removed by means of Ni—NTA affinity chromatography. The protein which had been purified in this way was separated by means of 1D PAGE and visualized with coomassie. The selectivity of the antiserum was confirmed in a Western blot after analogous separation of the recombinant protein.

1. An isolated regulatory CD4⁺ CD25⁺ T cell which contains at least one galectin.

2. An isolated T-regulatory cell as claimed in claim 1 which consists of the CD4⁺ CD25⁺β7⁺ subpopulation.

3. An isolated T-regulatory cell as claimed in claim 1 or 2 which contains at least one galectin selected from the group galectins 1-14.

4. An isolated T-regulatory cell as claimed in one of claims 1 to 3 which contains a human galectin or a homologous protein.

5. An isolated T-regulatory cell as claimed in one of claims 1 to 4 which contains at least one galectin selected from the group SEQ ID No. 1 to SEQ ID No. 5.

6. An isolated T-regulatory cell as claimed in one of claims 1 to 5 which contains at least one galectin selected from the group SEQ ID No. 1 or SEQ ID No. 2 having the isoforms: a.) apparent molecular weight of 14 kDa and a pI of 6.7, b.) apparent molecular weight of 13.5 kDa and a pI of 5.9, c.) apparent molecular weight of 13 kDa and a pI of 5.9.

7. An isolated T-regulatory cell as claimed in claim 6, wherein the isoforms are selected from the group SEQ ID No. 8 to SEQ ID No. 64.

8. An isolated regulatory T cell as claimed in one of claims 1 to 7, characterized in that at least one galectin is secreted, is located in the membrane or is presented on the surface of the T-regulatory cell or in the cytosol.

9. An isolated regulatory T cell as claimed in one of claims 1 to 8, characterized in that at least one galectin is concentrated in the regulatory T cell or on the surface of the regulatory T cell.

10. An isolated regulatory T cell as claimed in one of claims 1 to 9, characterized in that at least one nucleic acid encoding at least one galectin is present and, where appropriate, comprises one or more noncoding sequences and/or a poly(A) sequence and/or recognition sequences and/or regulatory sequences such as promoter or enhancer sequences.

11. An isolated T-regulatory cell as claimed in one of claims 1 to 9, characterized in that the nucleic acid sequence is selected from SEQ ID No. 6 or SEQ ID No. 7.

12. An isolated or native regulatory CD4⁺ CD25⁺ T cell which contains at least one galectin as target or marker.

13. An agent which binds at least one isolated regulatory T cell as claimed in one of claims 1-11 or native regulatory T cell as claimed in claim 12.

14. A binding agent as claimed in claim 13, which is selected from the group inhibitor, agonist, antagonist, probe, antibody or immunomodulator.

15. A binding agent as claimed in claim 13 or 14, wherein the binding agent exhibits one or more epitopes directed against galectin.

16. A binding agent as claimed in claim 15, wherein the binding agent additionally exhibits one or more epitopes directed against a surface protein.

17. A binding agent as claimed in claim 16, wherein the surface protein is selected from the group CD25, CD44, CD45, GITR, CTLA-4 and Fox P3.

18. A binding agent as claimed in one of claims 13 to 17, wherein the isolated regulatory T cell or native regulatory T cell containing at least one galectin is activated or inactivated.

19. A pharmaceutical which comprises at least one binding agent as claimed in one of claims 13 to 18 or isolated T-regulatory cells as claimed in one of claims 1 to 11.

20. A pharmaceutical as claimed in claim 19 for the treatment and therapy of diseases, specifically allergies, autoimmune diseases, in particular rheumatoid arthritis, multiple sclerosis or Crohn's disease, chronic inflammation, asthma, immunodeficiency diseases, AIDS, transplant rejection and cancer diseases as well as diabetes.

21. A pharmaceutical as claimed in claim 20, wherein the autoimmune diseases are selected from the group: alopecia greata, Bechterew's disease, antiphospholipid syndrome, Addison's disease, Behcet's disease, sprue celiac disease, chronic fatigue immune dysfunction syndrome (CFIDS), polyneuropathy, Churg-Strauss syndrome (granulomatosis), CREST syndrome (Raynaud's syndrome), cold agglutinin disease, cryoglobulinemia, fibromyalgia, fibromyositis, Basedow's disease, Guillain-Barré syndrome, idiopathic pulmonary fibrosis, idiopathic thrombocytopenia, IgA nephropathy, lichen planus, Ménière's disease, polyarteritis nodosa, polychondritis, polyglandular syndrome, polymyalgia rheumatica, primary agammaglobulinemia, biliary cirrhosis, psoriasis, Reiter's disease, sarcoidosis, Sjögren's disease, Takayasu arteritis, vasculitis, vitiligo and wegener's granulomatosis.

22. A test system which comprises at least one binding agent and at least one regulatory T cell containing galectins for identifying suitable binding agents or regulatory T cells, preferably those possessing elevated suppressive properties.

23. A test system which comprises at least one regulatory T cell containing galectins and at least one target cell, in particular T cell, B cell, macrophage, predendritic cell, dendritic cell, embryonic cell and/or fibroblast which is/are incubated with at least one regulatory T cell for the in-vitro detection of suppressive properties, in particular suppression of the cellular immune response of effector cells of the immune system, in particular B cells, NK cells, preferably T cells and T helper cells.

24. The test system as claimed in claim 23, wherein the effector cells are mammalian cells, in particular human or murine cells or an immune cell line and/or a cultured primary immune cell.

25. The test system as claimed in claim 23 or 24, wherein at least one further substance which can elicit an immune response, such as proteins, epitopes, protein fragments, antigens or binding agents, is/are incubated.

26. A diagnostic agent which comprises a test system as claimed in one of claims 22 to 25 and, where appropriate, a pharmaceutically acceptable support.

27. A diagnostic agent as claimed in claim 26 for diagnosing diseases, specifically allergies, autoimmune diseases, in particular rheumatoid arthritis, multiple sclerosis or Crohn's disease, chronic inflammation, asthma, immunodeficiency diseases, AIDS, transplant rejection and cancer diseases as well as diabetes.

28. A diagnostic agent as claimed in claim 27 for diagnosing diseases, specifically autoimmune diseases selected from the group: alopecia areata, Bechterew's disease, antiphospholipid syndrome, Addison's disease, Behcet's disease, sprue celiac disease, chronic fatigue immune dysfunction syndrome (CFIDS), polyneuropathy, Churg-Strauss syndrome (granulomatosis), CREST syndrome (Raynaud's syndrome), cold agglutinin disease, cryoglobulinemia, fibromyalgia, fibromyositis, Basedow's disease, Guillain-Barré syndrome, idiopathic pulmonary fibrosis, idiopathic thrombocytopenia, IgA nephropathy, lichen planus, Ménière's disease, polyarteritis nodosa, polychondritis, polyglandular syndrome, polymyalgia rheumatica, primary agammaglobulinemia, biliary cirrhosis, psoriasis, Reiter's disease, sarcoidosis, Sjögren's disease, Takayasu arteritis, vasculitis, vitiligo and Wegener's granulomatosis.
